SEQUENCE LISTING (92 sequences)

SEQ ID NO: 1 - protein, 9 aa, Influenza virus
YINTALLNA

SEQ ID NO: 2 - protein, 8 aa, Influenza virus
TVIKTNMI

SEQ ID NO: 3 - protein, 11 aa, Influenza virus
PVAGGTSSIYI

SEQ ID NO: 4 - protein, 9 aa, Influenza virus
MTIIFLILM

SEQ ID NO: 5 - protein, 9 aa, Influenza virus
ITFHGAKEI

SEQ ID NO: 6 - protein, 9 aa, Influenza virus
AIMDKNIIL

SEQ ID NO: 7 - protein, 9 aa, Influenza virus
AINGITNKV

SEQ ID NO: 8 - protein, 11 aa, Influenza virus
SPDDFALIVNA

SEQ ID NO: 9 - protein, 9 aa, Influenza virus
EEMGITTHF

SEQ ID NO: 10 - protein, 9 aa, Influenza virus
VETPIRNEW

SEQ ID NO: 11 - protein, 9 aa, Influenza virus
REILTKTTV

SEQ ID NO: 12 - protein, 9 aa, Influenza virus
LPFDRTTIM

SEQ ID NO: 13 - protein, 9 aa, Influenza virus
IIELAEKTM

SEQ ID NO: 14 - protein, 9 aa, Influenza virus
IIKLLPFTA

SEQ ID NO: 15 - protein, 10 aa, Influenza virus
ILDGVNGTLI

SEQ ID NO: 16 - protein, 8 aa, Influenza virus
IMNKSITL

SEQ ID NO: 17 - protein, 10 aa, Influenza virus
APPKQSRMQF

SEQ ID NO: 18 - protein, 9 aa, Influenza virus
ITNKVNTVI

SEQ ID NO: 19 - protein, 11 aa, Influenza virus
KIQRSQDPTML

SEQ ID NO: 20 - protein, 10 aa, Influenza virus
LTEKAVDSVT

SEQ ID NO: 21 - protein, 13 aa, Influenza virus
MIGIMPDMTPSTE

SEQ ID NO: 22 - protein, 10 aa, Influenza virus
MVIGMVSLVL

SEQ ID NO: 23 - protein, 10 aa, Influenza virus
NANTLSSVTT

SEQ ID NO: 24 - protein, 8 aa, Influenza virus
NIESRPQI

SEQ ID NO: 25 - protein, 10 aa, Influenza virus
NIVAEQGVTS

SEQ ID NO: 26 - protein, 11 aa, Influenza virus
NTDRGVTAACP

SEQ ID NO: 27 - protein, 10 aa, Influenza virus
PAATALATTI

SEQ ID NO: 28 - protein, 9 aa, Influenza virus
PMYVGVKSL

SEQ ID NO: 29 - protein, 11 aa, Influenza virus
QIMPCEPTIIE

SEQ ID NO: 30 - protein, 9 aa, Influenza virus
QVLAELKDI

SEQ ID NO: 31 - protein, 8 aa, Influenza virus
RVAMPKQI

SEQ ID NO: 32 - protein, 11 aa, Influenza virus
SIAEAIIVAMV

SEQ ID NO: 33 - protein, 11 aa, Influenza virus
STVASSLVLAV

SEQ ID NO: 34 - protein, 12 aa, Influenza virus
TADKRITEMIPE

SEQ ID NO: 35 - protein, 9 aa, Influenza virus
TIIAVSNIL

SEQ ID NO: 36 - protein, 9 aa, Influenza virus
VIAFAIVSI

SEQ ID NO: 37 - protein, 10 aa, Influenza virus
VILDVILHVV

SEQ ID NO: 38 - protein, 11 aa, Influenza virus
VTIGIASLILQ

SEQ ID NO: 39 - protein, 15 aa, Influenza virus
GTGSWPDGANINFMP

SEQ ID NO: 40 - protein, 14 aa, Influenza virus
GVSSACSYLENPSF

SEQ ID NO: 41 - protein, 14 aa, Influenza virus
IVWGIHHPATLKEH

SEQ ID NO: 42 - protein, 15 aa, Influenza virus
NTKLPFQNLSLRTVG

SEQ ID NO: 43 - protein, 10 aa, Influenza virus
QAAERMEVAS

SEQ ID NO: 44 - protein, 14 aa, Influenza virus
ITNKVNSIVDKMNT

SEQ ID NO: 45 - protein, 15 aa, Influenza virus
KESDEALNMTMASTP

SEQ ID NO: 46 - protein, 15 aa, Influenza virus
EESDEALKMSMASTP

SEQ ID NO: 47 - protein, 15 aa, Influenza virus
STQAAVDQINGKLNR

SEQ ID NO: 48 - protein, 9 aa, Influenza virus
LENERTLDF

SEQ ID NO: 49 - protein, 9 aa, Influenza virus
MEAVPLITI

SEQ ID NO: 50 - protein, 8 aa, Influenza virus
VEQEIRTF

SEQ ID NO: 51 - protein, 8 aa, Influenza virus
VEQELRTF

SEQ ID NO: 52 - protein, 8 aa, Influenza virus
YPDTGKVM

SEQ ID NO: 53 - protein, 8 aa, Influenza virus
YPDASKVM

SEQ ID NO: 54 - protein, 10 aa, Influenza virus
QPETCNQSII

SEQ ID NO: 55 - protein, 9 aa, Influenza virus
VPESKRMSL

SEQ ID NO: 56 - protein, 13 aa, Influenza virus
YAFAMSRGSGSGI

SEQ ID NO: 57 - protein, 9 aa, Influenza virus
WLTKSDGNS

SEQ ID NO: 58 - protein, 10 aa, Influenza virus
VTVACPYAGA

SEQ ID NO: 59 - protein, 10 aa, Influenza virus
TILLVITASN

SEQ ID NO: 60 - protein, 10 aa, Influenza virus
TVETANIGKI

SEQ ID NO: 61 - protein, 10 aa, Influenza virus
SQQRASAGQI

SEQ ID NO: 62 - protein, 9 aa, Influenza virus
SIFFESAGN

SEQ ID NO: 63 - protein, 10 aa, Influenza virus
SIGFYQILSI

SEQ ID NO: 64 - protein, 10 aa, Influenza virus
SLGIKSDAQL

SEQ ID NO: 65 - protein, 9 aa, Influenza virus
RVSETIQRF

SEQ ID NO: 66 - protein, 9 aa, Influenza virus
RVSETLQRF

SEQ ID NO: 67 - protein, 14 aa, Influenza virus
RTPGNAEIEDLIFL

SEQ ID NO: 68 - protein, 9 aa, Influenza virus
QAVAVVNYG

SEQ ID NO: 69 - protein, 9 aa, Influenza virus
MKTISLITI

SEQ ID NO: 70 - protein, 10 aa, Influenza virus
LLVALENQHI

SEQ ID NO: 71 - protein, 10 aa, Influenza virus
LLYALLATDA

SEQ ID NO: 72 - protein, 9 aa, Influenza virus
LMSELGVPF

SEQ ID NO: 73 - protein, 10 aa, Influenza virus
LNTSSRGILE

SEQ ID NO: 74 - protein, 8 aa, Influenza virus
KTFFGWKD

SEQ ID NO: 75 - protein, 9 aa, Influenza virus
KVKGQLKNN

SEQ ID NO: 76 - protein, 9 aa, Influenza virus
KVVKSVELN

SEQ ID NO: 77 - protein, 9 aa, Influenza virus
KVVQSVELN

SEQ ID NO: 78 - protein, 12 aa, Influenza virus
LANSKSQVNRQI

SEQ ID NO: 79 - protein, 13 aa, Influenza virus
ILRDCSVGGWLLG

SEQ ID NO: 80 - protein, 9 aa, Influenza virus
FAWRSINED

SEQ ID NO: 81 - protein, 10 aa, Influenza virus
DLLLKANSWS

SEQ ID NO: 82 - protein, 9 aa, Influenza virus
AIIVSMVFS

SEQ ID NO: 83 - protein, 13 aa, Influenza virus
LQSLQQIESIIEA

SEQ ID NO: 84 - protein, 12 aa, Influenza virus
ETKGVTAACSYS

SEQ ID NO: 85 - protein, 11 aa, Influenza virus
MQFGSSSEDLN

SEQ ID NO: 86 - protein, 13 aa, SARS coronavirus
QRGAEAAVKPLLA

SEQ ID NO: 87 - protein, 9 aa, SARS coronavirus
TRLQSLENV

SEQ ID NO: 88 - protein, 9 aa, SARS coronavirus
DVLSRLDKV

SEQ ID NO: 89 - protein, 9 aa, SARS coronavirus
LIIRENNTV

SEQ ID NO: 90 - protein, 11 aa, SARS coronavirus
FRVVMAMFSKT

SEQ ID NO: 91 - protein, 12 aa, SARS coronavirus
PMYTVSKGTQQS

SEQ ID NO: 92 - protein, 9 aa, SARS coronavirus
VTDVTQLYL

(19) United States
(12) Patent Application Publication
(10) Pub. No.: US 2014/0302124 A1
(43) Pub. Date: Oct. 9, 2014

(54) Cytotoxic T Lymphocyte Inducing Immunogens For Prevention Treatment and Diagnosis of INFLUENZA VIRUS INFECTION

(71) Applicant: Immunotope, inc., Doylestown, PA (US)

(72) Inventor: Ramila Philip, Ivyland, PA (US)

(73) Assignee: Immunotape, Inc., Doylestown, PA (US)

(21) Appl. No.: 14/352,089

(22) PCT Filed: Oct. 18, 2012
(86) PCT No.: PCT/US12/60734

§ 371 Date: Apr. 16, 2014

Related U.S. Application Data
(60) Provisional application No. 61/548,985, filed on Oct. 19, 2011.

(51) Int. Cl. C07K 7/06; A61K 39/215; A61K 45/06; C07K 7/08; A61K 39/145

(52) U.S. Cl. CPC C07K 7/06; C07K 7/08; A61K 39/145; A61K 45/06; A61K 39/215; A61K 2039/572
USPC 424/450; 530/328; 530/327; 530/326; 424/186.1; 424/85.2; 424/85.1; 435/5; 435/375

(57) ABSTRACT

Influenza virus infection and the resulting complications are a significant global public health problem and understanding the overall immune response to infection will contribute to appropriate management of the disease and its potentially severe complications. Improving humoral immunity to influenza is the target of current conventional influenza vaccines, however, these are generally not cross-protective. On the contrary, cell-mediated immunity generated by primary influenza infection provides substantial protection against serologically distinct viruses due to recognition of cross-reactive T cell epitopes, often from internal viral proteins conserved between viral subtypes. Efforts are underway to develop a universal flu vaccine that would stimulate both the humoral and cellular immune responses leading to long-lived memory. Such a universal vaccine should target conserved influenza virus antibody and T cell epitopes that do not vary from strain to strain. The present invention incorporates immunoproteomics to uncover novel MHC class I specific epitopes derived from influenza-infected cells. These epitopes are conserved with epitope-specific CTLs cross-reacting against various different influenza strains. These epitopes have potential as new informational and diagnostic tools to characterize T cell immunity in influenza infection, and serves as a universal vaccine candidate complementary to current vaccines.

### CROSS REFERENCE TO RELATED APPLICATIONS

This application is the US national application of PCT/US2012/060734, filed on 18 Oct. 2012, which claims priority to U.S. Provisional Application No. 61/548,985, filed Oct. 19, 2011, now expired, the disclosures of which are herein incorporated by reference in its entirety.

### FIELD OF THE INVENTION

The present invention relates generally to the field of immunogens whose structures incorporate polypeptides comprising epitopic peptides derived from proteins expressed by various strains of influenza virus infected cells and uses of said immunogens in eliciting cytotoxic T lymphocyte (CTL) responses for the diagnosis, prevention and treatment of multiple strains of influenza virus infection.

### BACKGROUND OF THE INVENTION

The mammalian immune system has evolved a variety of mechanisms to protect the host from microorganisms, an important component of this response being mediated by cells referred to as T cells and by antibodies derived from B cells. In combating bacterial infections, antibodies are especially important but likewise are specialized T cells that function primarily by recognizing and killing infected cells. The latter also function by secreting soluble molecules called cytokines that mediate a variety of functions of the immune system. Thus, the immune system is highly developed to deal with infectious organisms as well as with the elimination of cells infected with such organisms. Among the latter are viral infections, such as influenza virus infection.

Influenza virus is a significant public health problem internationally, causing three to five million cases of severe illness, and an estimated 250,000 to 500,000 deaths annually. Influenza virus is a member of orthomixovirdae and its genome is comprised of eight segments of negative single stranded RNA. Viral strains are divided into A, B, and C viruses and differ serologically only between the HA and NA proteins. Influenza constantly modifies these glycoproteins by implementing antigenic drift and shift, which is the main reason for influenza pandemics and the requirement for seasonal vaccines. The immune response to influenza is governed by both innate and adaptive immunity and has been well-studied. The humoral arm of the adaptive immune response utilizes secretory IgA and IgM to provide protection against the establishment of initial infection, while IgG acts to neutralize newly replicating virus. Improving humoral immunity to influenza is the target of current conventional influenza vaccines, however, they are generally not cross-protective. Cell-mediated immunity, on the other hand, as elicited by major histocompatibility complex (MHC) class I-restricted CD8+ cytotoxic T lymphocytes (CTLs), plays a central role in controlling influenza virus infection. Indeed, cell-mediated immunity generated by primary influenza infection provides substantial protection against serologically distinct viruses due to the recognition of cross-reactive epitopes, often from internal viral proteins conserved between viral subtypes.

Tremendous efforts are underway to develop a universal flu vaccine that would work against all types of influenza. Such a universal vaccine should target conserved influenza virus antibody and T cell epitopes that do not vary from strain to strain. Unfortunately, most conserved viral proteins lie within the virus, out of reach of antibodies. There is considerable evidence that T-cell responses are extremely important for protection against influenza. CD4+ T cells play a critical role in isotype-switching to IgG and in the generation of higher affinity antibodies and CTL memory. In humans, HA specific CD4+ T cells proliferate following influenza vaccination and aid the development of heterosubtypic influenza antibody responses. Importantly, in addition to the role of CTLs in mediating viral clearance, CD8+ CTLs in humans were shown to have cross-reactive responses to different subtypes of influenza A virus, thus playing a vital role in recovery from influenza virus infection, which may explain the relative paucity of disease among older, potentially vaccinated, or exposed individuals to H1N1 infection. Successful influenza vaccination campaigns can have enormous societal and economic impact.

The identification of peptides and proteins derived from influenza virus infection that are effectively recognized by the cellular arm of the immune response forms the basis for a new and effective vaccine. Such peptides are displayed on the surface of infected cells in association with MHC class I and class II molecules and serve as recognition targets for cytolytic and helper T cells of the immune system.

The present disclosure involves peptides that are associated with the HLA-A2, and HLA-B7 molecules, HLA-A2 supertypes, and HLA-B7 supertypes. A supertype is a group of HLA molecules that present at least one shared epitope. The present disclosure involves peptides that are associated with HLA molecules, and with the genes and proteins from which these peptides are derived.

Several methods have been developed to identify the peptides recognized by CTL, each method relying on the ability of a CTL to recognize and kill only those cells expressing the appropriate class I MHC molecule with the peptide bound to it. Such peptides can be derived from a non-self source, such as a pathogen (for example, following the infection of a cell by a virus, such as influenza virus infection) or from a self-derived protein within a cell, such as a cancerous cell.

Three different methodologies have typically been used for identifying the peptides that are recognized by CTLs in infectious disease field. These are: (1) the genetic method; (2) motif analysis; (3) the immunological and analytical chemistry methods or the Immunoproteomics method. The genetic and motif prediction methodologies have typically been used for identifying the peptides that are recognized by CTLs, which suffer from various drawbacks. A useful technique has been the immunoproteomics method involving a combination of cellular immunology and mass spectrometry. This approach involves the actual identification of endogenous CTL epitopes present on the cell surface by sequencing the naturally occurring peptides associated with class I MHC molecules. In this approach, cells are first lysed in a detergent solution, the peptides associated with the class I MHC molecules are purified, and the peptides are fractionated by high performance liquid chromatography (HPLC). Peptide sequencing is readily performed by tandem mass spectrometry. The sequence can be confirmed by direct synthesis thereof (See Examples 4 and 5, below). Once confirmed such synthetic peptides can be used to test their ability to activate CTLs against cells infected with the influenza virus.

A number of recent reports for different types of virus infections provide evidence that CTL specific for epitopes that are naturally processed and presented by infected cells have markedly greater impact on the control of virus replication. Undoubtedly, CTLs have been shown to play an important role in the elimination of influenza virus-infected cells. Thus, identification of antigenic peptides that are presented by infected cells and recognized by epitope-specific CTLs may suggest new ways to suppress viral replication and prevent persistent infection. Multiple peptides from conserved regions of influenza virus may prove essential in the development of a universally immunogenic vaccine.

Little is known about cross strain conserved T cell epitopes that are immunologically relevant in eliciting an effective T cell response to the various influenza strains. Several groups have attempted to identify T cell epitopes by either motif prediction of MHC binding peptides from influenza proteins, or by screening overlapping peptides from structural and nonstructural viral proteins. Screening PBMCs from infected individuals using a panel of algorithm-derived peptide sequences identified a few cross strain specific T cell epitopes. However, a comprehensive analysis of naturally presented epitopes on infected cells has never been undertaken or reported.

### SUMMARY OF THE INVENTION

The present invention relates to immunogens comprising polypeptides with amino acid sequences comprising epitopic sequences selected from the sequences of SEQ ID NO: 1-92 and which immunogens facilitate a cytotoxic T lymphocyte (CTL)-mediated immune response against various strains of influenza virus infected cells.

The present invention also relates to nucleic acid molecules that encode polypeptides comprising said epitopic peptide, and which can also be used to facilitate an immune response against influenza infected cells.

The present invention provides compositions comprising the polypeptides and immunogens described herein whereby the oligopeptides and polypeptides of such immunogens are capable of inducing a CTL response against cells expressing a protein comprising an epitopic sequence of SEQ ID NO: 1-92 presented in association with Class I MHC protein, which cells are infected with various strains of influenza virus.

In specific embodiments, the oligopeptides of the invention have a sequence that comprises SEQ ID NO: 1-92 and are used as part of a larger structure, most advantageously a polypeptide, including both naturally occurring polypeptides and synthetic polypeptides. The immunogens of the invention incorporate such epitopic peptide sequences, either with such sequences attached to form a larger antigenic structure or just as part of a polypeptide sequence incorporating such peptides as part of the amino acid sequence thereof.

Where the immunogens of the invention are polypeptides, or mixtures of polypeptides, such polypeptides can be of any length as long as part of their sequence comprises at least one peptide of SEQ ID NO: 1-92, or sequence highly homologous thereto, ordinarily differing by no more than one amino acid residue, including multiple copies of said sequence, when it is desired to induce a CTL response against such peptide and thereby against influenza virus infected cells.

The present invention further relates to polynucleotides comprising the gene coding for a polypeptide of the immunogens disclosed herein. The present invention also provides methods that comprise contacting a lymphocyte, especially a CTL, with an immunogen or its isoforms or splice variants of the invention under conditions that induce a CTL response against various strains of influenza virus infected cell, and more specifically influenza virus A infected cell. The methods may involve contacting the CTL with the immunogenic peptide in vivo, in which case the peptides, polypeptides, and polynucleotides of the invention are used as vaccines, and will be delivered as a pharmaceutical composition comprising a pharmaceutically acceptable carrier or delivery system and the immunogen, typically along with an adjuvant or one or more cytokines.

Alternatively, the immunogens of the present invention can be used to induce a CTL response in vitro. The generated CTL can then be introduced into a patient with influenza virus infection. Alternatively, the ability to generate CTLs in vitro can serve as a diagnostic for influenza virus infection.

### BRIEF DESCRIPTION OF THE DRAWINGS

FIG. 1: Influenza strain infection analysis. HepG2, JY, and monocyte-derived human DC were pulsed with 1000 HAU/1×10⁶ cells of purified virus (PR8, X31) or 200 HAU/1×10⁶ cells of unpurified virus (JAP) in PBS+0.1% BSA for approximately 1 hr at 37° C. Cells were fixed and intracellularly stained for influenza NP after overnight incubation and analyzed in a flow cytometry. The peak shift indicates the increase in mean fluorescence due to infection.

FIG. 2: CTLs generated in vitro with influenza T cell epitopes are specific and cross-reactive. (A) HepG2 and JY cells were left untreated or infected with PR8 and used as targets in an ELISpot assay with CTLs that were generated from HLA-A2+ PBMCs against specific peptides. (B) HepG2 cells were infected with PR8, X-31, or JAP and used as targets in an ELISpot assay. Results were normalized against uninfected controls.

FIG. 3: CTLs generated with influenza T cell epitopes in vivo using human HLA-A2 transgenic mice are specific and cross-reactive. (A) Immunization scheme for peptide injections. (B) T2 cells were pulsed with peptide and used as targets in an ELISpot assay with CTLs that were generated from humanized mice immunized with influenza-specific (P1-5) peptides. (C) HepG2 and JY cells were infected with PR8, X-31, or JAP and used as targets in an ELISpot assay. (D) Additionally, cells were stained for CD8 and CD107a after overnight co-culture and results are given as the mean fluorescence intensity of CD107a gated on CD8+ cells. Results were normalized against unpulsed or uninfected controls. FIG. 4: CTLs generated in vivo with influenza T cell epitopes combined with a cross strain shared antibody epitope (pM2e) are specific and cross-reactive. HepG2 and JY cells were infected with PR8, X-31, or JAP and used as targets in an (A) ELISpot assay or (B) FACS analysis using CTLs generated from humanized mice immunized with influenza-specific (P1-5) peptides plus pM2e. (C) Serum titers of mice immunized with PBS, P1-5+pM2e, or pM2e alone were analyzed using ELISA. Plates were coated with pM2e and serum was added and serially diluted. Serum IgG was then detected using anti-mouse IgG secondary reagents. The dilution where the signal was reduced to background was measured and graphed using the reciprocal of the dilution factor (1/DF).

### DETAILED DESCRIPTION OF THE INVENTION

### Definitions

As used herein and except as noted otherwise, all terms are defined as given below. The term “peptide” is used herein to designate a series of amino acid residues, connected one to the other typically by peptide bonds between the alpha-amino and carbonyl groups of the adjacent amino acids. The peptides are typically 9 amino acids in length, but can be as short as 8 amino acids in length, and as long as 14 amino acids in length. The series of amino acids are consider an “oligopeptide” when the amino acid length is greater than about 14 amino acids in length, typically up to about 30 to 40 residues in length. When the amino acid residue length exceeds 40 amino acid residues, the series of amino acid residues is termed “polypeptide”.

A peptide, oligopeptide, polypeptide, protein, or polynucleotide coding for such a molecule is “immunogenic” and thus an immunogen within the present invention if it is capable of inducing an immune response. In the present invention, immunogenicity is more specifically defined as the ability to induce a CTL-mediated response. Thus, an immunogen would be a molecule that is capable of inducing an immune response, and in the present invention, a molecule capable of inducing a CTL response. An immunogen may have one or more isoforms or splice variants that have equivalent biological and immunological activity, and are thus also considered for the purposes of this invention to be immunogenic equivalents of the original, natural polypeptide.

A T cell “epitope” is a short peptide molecule that binds to a class I or II MHC molecule and that is subsequently recognized by a T cell. T cell epitopes that bind to class I MHC molecules are typically 8-14 amino acids in length, and most typically 9 amino acids in length.

Three different genetic loci encode for class I MHC molecules: HLA-A, HLA-B, and HLA-C. The present invention involves peptides that are associated with HLA-A2 supertypes. A supertype is a group of HLA molecules that present at least one shared epitope. MHC molecule peptides that have been found to bind to one member of the MHC allele supertype family (A2 for example) are thought to be likely to bind to other members of the same supertype family (A68 for example).

As used herein, reference to a DNA sequence includes both single stranded and double stranded DNA. Thus, the specific sequence, unless the context indicates otherwise, refers to the single strand DNA of such sequence, the duplex of such sequence with its complement (double stranded DNA) and the complement of such sequence.

The term “nucleotide sequence” refers to a heteropolymer of deoxyribonucleotides. The nucleotide sequence encoding for a particular peptide, oligopeptide, or polypeptide naturally occurring or synthetically constructed.

The term “fragment,” when referring to a coding sequence, means a portion of DNA comprising less than the complete coding region whose expression product retains essentially the same biological or immunological function or activity as the expression product of the complete coding region.

The term “isolated” means that the material is removed from its original environment (e.g., the natural environment if it is naturally occurring).

The polynucleotides, and recombinant or immunogenic polypeptides, disclosed in accordance with the present invention may also be in “purified” form.

The term “active fragment” means a fragment that generates an immune response (i.e., has immunogenic activity) when administered, alone or optionally with a suitable adjuvant, to an animal, such as a mammal, for example, a human, such immune response taking the form of stimulating a CTL response within the recipient. Alternatively, the “active fragment” may also be used to induce a CTL response in vitro.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, the oligopeptides resulting from such treatment would represent portions, segments or fragments of the starting polypeptide.

The term “percent identity” when referring to a sequence, means that a sequence is compared to a described sequence after alignment of the sequence to be compared with the described sequence. The Percent Identity is determined according to the following formula:

Percent Identity=100 [1−(C/R)]

wherein C is the number of differences between the Reference Sequence (“R”) and the Compared Sequence (“C”) over the length of alignment between R and C wherein (i) each base or amino acid in R that does not have a corresponding aligned base or amino acid in the C and (ii) each gap in R and (iii) each aligned base or amino acid in R that is different from an aligned base or amino acid in C, constitutes a difference; and R is the number of bases or amino acids over the length of the alignment with C with any gap created in R also being counted as a base or amino acid.

### Description

The present invention relates generally to immunogens and immunogenic compositions, and methods of use thereof, for the prevention, treatment, and diagnosis of influenza viral infections, especially influenza A virus infection. Disclosed according to the invention are immunogens comprising proteins or polypeptides whose amino acid sequences comprise one or more epitopic peptides with sequences homologous to, preferably identical to, the sequence of SEQ ID NO: 1-92. In addition, the invention further relates to polynucleotides that can be used to stimulate a CTL response against influenza virus-infected cells, especially cells infected with the causative organism of influenza A virus.

One embodiment of the present invention includes compositions for influenza peptides, subsequence and portions thereof, nucleic acid sequences encoding influenza peptides, subsequences and portions thereof, and host cells expressing influenza peptides, subsequences and portions thereof. One particular aspect of the subsequence or portion of the influenza polypeptide sequence includes epitopic peptides. These embodiments further incorporate useful pharmaceutical compositions such as, but not limited to, an adjuvant (e.g., Freund' s complete or incomplete adjuvant) or administration with traditional prophylactic viral vaccine formulations (e.g., live attenuated viruses, inactivated viruses, recombinant proteins, chimeric viruses, DNA vaccines, and synthetic peptides).

The invention includes kits that contain influenza peptides, subsequences and portions thereof, compositions, that optionally include instructions for treating (prophylactic or therapeutic), vaccinating or immunizing a subject against an influenza infection, or treating (prophylactic or therapeutic) a subject having or at risk of having an influenza virus infection or pathology.

In accordance with further embodiments of the invention, methods for treating a subject having an influenza infection (acute) are provided. In one embodiment, a method includes administering to a subject in need thereof an amount of influenza peptide or epitopic peptide, subsequence or portion thereof, sufficient to treat the subject for the pathogen infection.

In accordance with further embodiments of the invention, there are provided prophylactic methods including methods of vaccinating and immunizing a subject against an influenza infection (acute) such as, but not limited to, protecting a subject against influenza infection to decrease or reduce the probability of an influenza infection or pathology in a subject or to decrease or reduce susceptibility of a subject to an influenza infection or pathology or to inhibit or prevent influenza infection in a subject.

In accordance with further embodiments of the present invention specific oligopeptide sequences are disclosed with amino acid sequences shown in SEQ ID NO: 1-92 representing epitopic peptides (i e immunogenic oligopeptide sequences) of at least about 8 amino acids in length, preferably about 9 amino acids in length (i.e., nonapeptides), and no longer than about 14 amino acids in length and present as part of a larger structure, such as a polypeptide or full length protein.

The polypeptides forming the immunogens of the present invention have amino acid sequences that comprise at least one stretch, possibly two, or more stretches of about 8 to 10 or up to 14 residues in length and which stretches differ in amino acid sequence from the sequences of SEQ ID NO: 1-92 by no more than about 1 amino acid residue, preferably a conservative amino acid residue, especially amino acids of the same general chemical character, such as where they are hydrophobic amino acids.

These polypeptides are of any desired length so long as they have immunogenic activity in that they are able, under a given set of desirable conditions, to elicit in vitro or in vivo the activation of cytotoxic T lymphocytes (CTLs) (i.e., a CTL response) against a presentation of various strains of influenza specific protein, where said proteins are presented in vitro or in vivo by an antigen presenting cell (APC). The proteins and polypeptides forming the immunogens of the present invention can be naturally occurring or synthesized chemically.

The present invention further embodies an isolated polypeptide, especially one having immunogenic activity, the sequence of which comprises within it one or more stretches comprising any 2 or more of the sequences of SEQ ID NO: 1-92 and in any relative quantities and wherein said sequences may differ by one amino acid residues from the sequences of SEQ ID NO: 1-92 in any given stretch of 8 to 10, or up to 14 amino acid residues. Thus within the present invention, by way of a non-limiting example only, such polypeptide may contain as part of its amino acid sequence, nonapeptide fragments having up to 8 amino acids identical to a sequence of SEQ ID NO: 1,2,7,8 such that the polypeptide comprises, in a specific embodiment, 2 segments with at least 8 residues identical to SEQ ID NO: 1 and SEQ ID NO: 2 and one segment with at least 8 residues identical to SEQ ID NO: 7. In other embodiments, other combinations and permutations of the epitopic sequences disclosed herein may be part of an immunogen of the present invention or of such a polypeptide so long as any such polypeptide comprises at least 2 such epitopes, whether such epitopes are different or the same.

All of the epitopic peptides of SEQ ID NO: 1 through 85 are derived from proteins expressed by DV infected cells and sequences and were identified through the method of Immunoproteomics and Automated High Through-put Sequencing (HTPS).

In addition to the sequences of SEQ ID NO: 1-92, the proteins and polypeptides forming the immunogens of the present invention further comprise one or more other immunogenic amino acid stretches known to be associated with influenza infection, and more specifically multiple stains of influenza, and which may stimulate a CTL response whereby the immunogenic peptides associate with HLA-A2 or HLA-A24 or HLA-B7, HLA supertypes, or any class I MHC (i.e., MHC-1) molecule.

The immunogens of the present invention can be in the form of a composition of one or more of the different immunogens and wherein each immunogen is present in any desired relative abundance.

The oligopeptides and polypeptides useful in practicing the present invention may be derived by fractionation of naturally occurring proteins by methods such as protease treatment, or they may be produced by recombinant or synthetic methodologies that are well known and clear to the skilled artisan. The polypeptide may comprise a recombinant or synthetic polypeptide having at least one of SEQ ID NO: 1-92. Thus, oligopeptides and polypeptides of the present invention have at least one immunogenic peptides within the amino acid sequence of said oligopeptides and polypeptides, and said immunogenic peptides, or epitopes, which are the same or different, or may have any number of such sequences wherein some of them are identical to each other in amino acid sequence and said epitopic sequences occur in any order within said immunogenic polypeptide sequence. The location of such sequences within the sequence of a polypeptide forming an immunogen may affect relative immunogenic activity. In addition, immunogens of the present invention may comprise more than one protein comprising the amino acid sequences disclosed herein. Such polypeptides may be part of a single composition or may themselves be covalently or non-covalently linked to each other.

The immunogenic peptides disclosed herein may also be linked directly to, or through a spacer or linker to: an immunogenic carrier such as serum albumin, tetanus toxoid, keyhole limpet hemocyanin, dextran, or a recombinant virus particle; an immunogenic peptide known to stimulate a T helper cell type immune response; a cytokine such as interferon gamma or GMCSF; a targeting agent such as an antibody or receptor ligand; a stabilizing agent such as a lipid; or a conjugate of a plurality of epitopes to a branched lysine core structure, such as the so-called “multiple antigenic peptide” described by Posenett et. al.; a compound such as polyethylene glycol to increase the half-life of the peptide; or additional amino acids such as a leader or secretory sequence, or a sequence employed for the purification of the mature sequence. Spacers and linkers typically comprise relatively small, neutral molecules. In addition, such linkers need not be composed of amino acids but any oligomeric structures will do as well so long as they provide the correct spacing so as to optimize the desired level of immunogenic activity of the immunogens of the present invention. The immunogen may therefore take any form that is capable of eliciting a CTL response.

Immunogens, such as proteins, oligopeptides and polypeptides of the invention, are structures that contain the peptides disclosed according to the present invention but such immunogenic peptides may not necessarily be attached thereto by the conventional means of using ordinary peptide bounds. The immunogens of the present invention simply contain such peptides as part of their makeup, but how such peptides are to be combined to form the final immunogen is through any means known in the art.

The peptides that are naturally processed and bound to a class I MHC molecule, and which are recognized by the DV-specific CTL, need not be the optimal peptides for stimulating a CTL response. Thus, the ability to modify a peptide such that it more readily induces a CTL response is considered. Generally, the peptides may be modified at amino acid residues that are predicted to interact with the class I MHC molecule, in which case the goal is to create a peptide that has a higher affinity for the class I MHC molecule than does the original peptide. The peptides can be modified at amino acid residues that are predicted to interact with the T cell receptor on the CTL, in which case the goal is to create a peptide that has a higher affinity for the T cell receptor than does the original peptide. Both of these types of modifications can result in a variant peptide that is related to an original peptide, but which is better able to induce a CTL response than is the original peptide as selected from SEQ ID NO: 1-92.

The original peptides disclosed herein can be further modified by the substitution of one or more residues at different, possibly selective, sites within the peptide chain. Such substitutions can be conservative. Less conservative substitutions or even highly non-conservative replacements are also considered since chemical effects are not totally predictable.

Based on cytotoxicity assays, an epitope is considered substantially identical to the reference peptide if it has at least 10% of the antigenic activity of the reference peptide as defined by the ability of the substituted peptide to reconstitute the epitope recognized by a CTL in comparison to the reference peptide. Thus, when comparing the lytic activity in the linear portion of the effector:target curves with equimolar concentrations of the reference and substituted peptides, the observed percent specific killing of the target cells incubated with the substituted peptide should be equal to that of the reference peptide at an effector:target ratio that is no greater than 10-fold above the reference peptide effector:target ratio at which the comparison is being made.

Preferably, when the CTLs specific for a peptide of SEQ ID NO: 1-92 are tested against the substituted peptides, the peptide concentration at which the substituted peptides achieve half the maximal increase in lysis relative to background is no more than about 1 mM, preferably no more than about 1 pM, more preferably no more than about 1 nM, and still more preferably no more than about 100 pM, and most preferably no more than about 10 pM. It is also preferred that the substituted peptide be recognized by CTLs from at least two or more individuals, preferably three.

Thus, the epitopes of the present invention may be identical to naturally occurring DV infected cell-associated or DV-specific epitopes or may include epitopes that differ by no more than 4 residues from the reference peptide, as long as they have substantially identical antigenic activity.

It should be appreciated that an immunogen may comprise one or more peptides from SEQ ID NO: 1-92, a plurality of peptides selected from SEQ ID NO: 1-92, or comprise a polypeptide that itself comprises one or more of the epitopic peptides of SEQ ID NO: 1-92.

The immunogenic peptides and polypeptides of the invention can be prepared synthetically, or any means known in the art, including those techniques involving recombinant DNA technology.

The coding sequences for peptides of the length contemplated herein can be synthesized on commercially available automated DNA synthesizers or modified to a desired amino acid substitution. The coding sequence can be transformed or transfected into suitable hosts to produce the desired fusion protein.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation. Such cells can routinely be utilized for assaying CTL activity by having said genetically engineered, or recombinant, host cells express the immunogenic peptides of the present invention.

The immunogenic peptides of the present invention may be used to elicit CTLs ex vivo from either healthy individuals or from influenza infected individuals. Such responses are induced by incubating in tissue culture the individual's CTL precursor lymphocytes together with a source of antigen presenting cells and the appropriate immunogenic peptide. The CTLs generated with peptides and polypeptides of the invention can be prepared by any means known in the art, including those techniques involving ex vivo adoptive cell therapy technologies.

A variety of approaches are known in the art that allow polynucleotides to be introduced and expressed in a cell, thus providing one or more peptides of the invention to the class I MHC molecule binding pathway. Oligonucleotides that code for one or more of the peptides of the invention can be provided to antigen presenting cells in such a fashion that the peptides associate with class I MHC molecules and are presented on the surface of the antigen presenting cell, and consequently are available to stimulate a CTL response.

By preparing the stimulator cells used to generate an in vitro CTL response in different ways, it is possible to control the peptide specificity of CTL response. For example, the CTLs generated with a particular peptide will necessarily be specific for that peptide. Likewise, CTLs that are generated with a polypeptide or polynucleotide expressing or coding for particular peptides will be limited to specificities that recognize those peptides. More broadly, stimulator cells, and more specifically dendritic cells, can be incubated in the presence of the whole parent protein. As a further alternative, stimulator cells, and more specifically dendritic cells, can be transduced or transfected with RNA or DNA comprising the polynucleotide sequence encoding the protein. Under these alternative conditions, peptide epitopes that are naturally cleaved out of the protein, and which are generated in addition to peptide epitopes of SEQ ID NO:1-92 can associate with an appropriate class I MHC molecule, which may or may not include HLA-A1, -A2, -A24, -B7. The selection of antigen presenting cells and the type of antigen with which to stimulate the CTL, is left to the ordinary skilled artisan.

In certain embodiments, the methods of the present invention include a method for inducing a CTL response in vitro that is specific for influenza infected cell expressing a molecule from A1, A2, A24, or B7 supertypes, whereby the method comprises contacting a CTL precursor lymphocyte with an antigen presenting cell that has bound an immunogen or has exogenously acquired an immunogenic oligopeptide or polypeptide comprising one or more of the peptides disclosed according to the invention.

Another embodiment is directed to a process for inducing a CTL response in vitro that is specific for influenza infected cell expressing a molecule from A1, A2, A24, or B7 supertypes, comprising contacting a CTL precursor lymphocyte with an antigen presenting cell that is expressing a polynucleotide coding for a polypeptide of the invention and wherein said polynucleotide is operably linked to a promoter.

A variety of techniques exist for assaying the activity of CTL. Such assays are well-known in the art and their selection is left to the skilled artisan. CTLs are known to release, induce, increase, enhance, stimulate or activate expression or production of a cytokine. Assay selection is left to the skilled artisan.

After expansion of the antigen-specific CTLs, the CTLs are then adoptively transferred back into the patient, where they will destroy their specific target cell. Methodologies for reinfusing T cells into a patient are well known and exemplified in U.S. Pat. No. 4,844,893 to Honski, et al., and U.S. Pat. No. 4,690,915 to Rosenberg.

The peptide-specific CTL can be purified from the stimulator cells prior to infusion into the patient. For example, monoclonal antibodies directed toward the cell surface protein CD8, present on CTL, can be used in conjunction with a variety of isolation techniques such as antibody panning, flow cytometric sorting, and magnetic bead separation to purify the peptide-specific CTL away from any remaining non-peptide specific lymphocytes or from the stimulator cells.

Thus, one embodiment of the present invention relates to a process for treating a subject with DV infection or influenza exposure characterized by infected cells expressing complexes of a molecule from A2, or A24, or B7 supertypes, whereby CTLs produced in vitro according to the present invention are administered in an amount sufficient to destroy the infected cells through direct lysis or to effect the destruction of the infected cells indirectly through the elaboration of cytokines.

Another embodiment of the present invention is directed to a process for treating a subject with infection characterized by infected cells expressing any class I MHC molecule and an epitope of SEQ ID NO: 1-92, whereby the CTLs are produced in vitro and are specific for the epitope or original protein and are administered in an amount sufficient to destroy the infected cells through direct lysis or to effect the destruction of the infected cells indirectly through the elaboration of cytokines.

The ex vivo generated CTL can be used to identify and isolate the T cell receptor molecules specific for the peptide. The genes encoding the alpha and beta chains of the T cell receptor can be cloned into an expression vector system and transferred and expressed in naive T cells from peripheral blood, T cells from lymph nodes, or T lymphocyte progenitor cells from bone marrow. These T cells, which would then be expressing a peptide-specific T cell receptor, would then have anti-influenza reactivity and could be used in adoptive therapy of influenza infection, and more specifically multiple strains of influenza.

In addition to their use for therapeutic or prophylactic purposes, the immunogenic peptides of the present invention are useful as screening and diagnostic agents. Thus, the immunogenic peptides of the present invention, together with modern techniques of CTL screening, make it possible to screen patients for the presence of T cells specific for these peptides as a test for influenza infection, exposure and immune response. The results of such screening may help determine the efficacy of proceeding with the regimen of treatment disclosed herein using the immunogens of the present invention.

The oligopeptides of the invention, such as SEQ ID NO: 1-92, can also be used to prepare class I MHC tetramers or pentamers or dextramers which can be used in conjunction with flow cytometry to quantitate the frequency of peptide-specific CTL that are present in a sample of lymphocytes from an individual. Specifically, for example, class I MHC molecules comprising peptides of SEQ ID NO: 1-92, would be combined to form tetramers as exemplified in U.S. Pat. No. 5,635,363. Said tetramers would find use in monitoring the frequency of CTLs in the peripheral blood or lymph nodes of an individual who is vaccinated or undergoing immunotherapy with the peptides, proteins, or polynucleotides of the invention, and it would be expected that successful immunization would lead to an increase in the frequency of the peptide-specific CTL.

Alternatively, the immunogenic peptides disclosed herein, as well as functionally similar homologs thereof, may be used to screen a sample for the presence of CTLs that specifically recognize the corresponding epitopes. The lymphocytes to be screened in this assay will normally be obtained from the peripheral blood, but lymphocytes can be obtained from other sources, including lymph nodes, spleen, and body fluids. The peptides of the present invention may then be used as a diagnostic tool to evaluate the efficacy of the immunotherapeutic treatments disclosed herein. Thus, the in vitro generation of CTL as described above would be used to determine if patients are likely to respond to the peptide in vivo. Similarly, the in vitro generation of CTL cam be done with samples of lymphocytes obtained from the patient before and after treatment with the peptides. Successful generation of CTL in vivo should then be recognized by a correspondingly easier ability to generate peptide-specific CTL in vitro from lymphocytes obtained following treatment in comparison to those obtained before treatment.

As stated above, a prophylactic or therapeutic vaccine in accordance with the present invention may include one or more of the hereinabove described polypeptides or active fragments thereof, or a composition, or pool, of immunogenic peptides disclosed herein. When employing more than one polypeptide or active fragment, such as two or more polypeptides and/or active fragments may be used as a physical mixture or as a fusion of two or more polypeptides or active fragments. The fusion fragment or fusion polypeptide may be produced, for example, by recombinant techniques or by the use of appropriate linkers for fusing previously prepared polypeptides or active fragments.

The immunogenic molecules of the invention, including vaccine compositions, may be utilized according to the present invention for purposes of preventing, suppressing or treating diseases causing the expression of the immunogenic peptides disclosed herein, such as where the antigen is being expressed by influenza infected cells. As used in accordance with the present invention, the term “prevention” relates to a process of prophylaxis in which an animal, especially a mammal, and most especially a human, is exposed to an immunogen of the present invention prior to the induction or onset of the disease process. This could be done where an individual is at high risk for any influenza infection based on the living or travel to the influenza pandemic areas. Alternatively, the immunogen could be administered to the general population as is frequently done for any infectious diseases. Alternatively, the term “suppression” is often used to describe a condition wherein the disease process has already begun but obvious symptoms of said condition have yet to be realized. Thus, the cells of an individual may have been infected but no outside signs of the disease have yet been clinically recognized. In either case, the term prophylaxis can be applied to encompass both prevention and suppression. Conversely, the term “treatment” is often utilized to mean the clinical application of agents to combat an already existing condition whose clinical presentation has already been realized in a patient. This would occur where an individual has already been diagnosed as having confirmed influenza infection.

It is understood that the suitable dosage of an immunogen of the present invention will depend upon the age, sex, health, and weight of the recipient, the kind of concurrent treatment, if any, the frequency of treatment, and the nature of the effect desired. However, the most preferred dosage can be tailored to the individual subject, as determined by the researcher or clinician. The total dose required for any given treatment will commonly be determined with respect to a standard reference dose as set by a manufacturer, such as is commonly done with vaccines, such dose being administered either in a single treatment or in a series of doses, the success of which will depend on the production of a desired immunological result (i.e., successful production of a CTL-mediated response to the antigen, which response gives rise to the prevention and/or treatment desired).

The therapeutically effective amount of a composition containing one or more of the immunogens of this invention, is an amount sufficient to induce an effective CTL response to prevent, cure or arrest disease progression. Thus, this dose will depend, among other things, on the identity of the immunogens used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Thus, for purposes of prophylactic or therapeutic administration, effective amounts would generally lie within the range of from 1.0 μg to about 5,000 μg of peptide for a 70 kg patient, followed by boosting dosages of from about 1.0 μg to about 1,000 μg of peptide pursuant to a boosting regimen over days, weeks or months, depending on the recipient's response and as necessitated by subsequent monitoring of CTL-mediated activity within the bloodstream. Of course, such dosages are to be considered only a general guide and, in a given situation, may greatly exceed such suggested dosage regimens where the clinician believes that the recipient's condition warrants more aggressive administration schedule. The efficacy of administering additional doses, and of increasing or decreasing the interval, may be re-evaluated on a continuing basis, in view of the recipient's immunocompetence (for example, the level of CTL activity with respect to acute or chronic influenza infection).

For such purposes, the immunogenic compositions according to the present invention may be used against a influenza infection by administration to an individual by a variety of routes. The composition may be administered parenterally or orally, and, if parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time.

Generally, vaccines are prepared as injectables, in the form of aqueous solutions or suspensions. Pharmaceutical carriers, diluents and excipients can be generally added that are compatible with the active ingredients and acceptable for pharmaceutical use.

The concentration of the CTL stimulatory peptides of the invention in pharmaceutical formulations are subject to wide variation, including anywhere from less than 0.01% by weight to as much as 50% or more. Factors such as volume and viscosity of the resulting composition must also be considered. The solvents, or diluents, used for such compositions include water, dimethylsulfoxide, PBS (phosphate buffered saline), or saline itself, or other possible carriers or excipients.

Aerosol administration is also an alternative, requiring only that the immunogens be properly dispersed within the aerosol propellant. The use of a surfactant to properly disperse the immunogen may be required. Representative surfactants include caproic, octanoic, lauric, palmitic, stearic, linoleic, linolenic, olesteric and oleic acids with an aliphatic polyhydric alcohol or its cyclic anhydride. The surfactant may constitute 0.1-20% by weight of the composition, preferably 0.25-5%.

The peptides and polypeptides of the invention may also be delivered with an adjuvant. Adjuvant effects can also be obtained by injecting a variety of cytokines along with the immunogens of the invention.

The peptides and polypeptides of the invention can also be added to professional antigen presenting cells such as dendritic cells that have been prepared ex vivo.

The present invention is also directed to a vaccine in which an immunogen of the present invention is delivered or administered in the form of a polynucleotide encoding a polypeptide or active fragment as disclosed herein, whereby the peptide or polypeptide or active fragment is produced in vivo. The polynucleotide may be included in a suitable expression vector and combined with a pharmaceutically acceptable carrier. A wide variety of vectors are available and apparent to those skilled in the art. Vaccinia vectors and methods useful in immunization protocols are described in U.S. Pat. No. 4,722,848, the disclosure of which is incorporated herein by reference in its entirety.

Regardless of the nature of the composition given, additional vaccine compositions may also accompany the immunogens of the present invention. Thus, for purposes of preventing or treating DV infection (e.g., prophylactic or therapeutic vaccine), compositions containing the immunogens disclosed herein may, in addition, contain other vaccine pharmaceuticals. The use of such compositions with multiple active ingredients is left to the discretion of the clinician.

While examples are provided to illustrate the invention, it is to be understood that these examples in no way limit the invention to the embodiments described herein and that other embodiments and uses will no doubt suggest themselves to those skilled in the art. All publications, patents, and patent applications cited herein are hereby incorporated by reference, as are the references cited therein. It is also to be understood that throughout this disclosure where the singular is used, the plural may be inferred and vice versa and use of either is not to be considered limiting.

### EXAMPLE

Influenza A and B viral strains (A/New Caledonia/20/99 (H1N1), A/Wisconsin/67/2005 (H3N2), B/Malaysia/2506/2004) were obtained from Charles River Laboratories. HepG2, hepatoma cells, JY, EBV transformed lymphoblastoid B cells, and T2, lymphoblasts were obtained from ATCC. HepG2 were maintained in DMEM:F12 medium while JY and T2, were maintained in RPMI 1640 (Mediatech, Manassas, Va.) supplemented with 10% fetal bovine serum, and maintained at 37° C. in a humidified incubator with 5% CO2. Dendritic cells (DC) were generated from leukopheresis obtained from HLA-A2+ healthy donors (Research Blood Components, LLC, Brighton, Mass.) and processed as described previously (James S. Testa, et al. (2012), PLoSOne in press). HepG2, JY cells and fresh human DCs were infected with purified influenza vaccine strain at 100 HAU per 10⁶ cells. Twenty-four hr after infection, cells were harvested, washed two times in phosphate buffered saline (pH 7.4) and cell pellets stored at −80.degree. C.

Cell lysates were prepared from influenza infected cells and MHC/peptide complexes were isolated by immunoaffinity chromatography using MHC molecule specific antibodies The peptides purified from the MHC molecules were fractionated using C-18 reversed phase (RP) column (4 6mm diameterx150 mm length) using an offline HPLC (Dionex, Sunnyvale, Calif.). The peptide containing fractions were collected and dried to 6 μL under vacuum for LC/MS/MS analysis.

Mass spectrometry experiments were carried out using LTQ (Thermo) and Orbitrap instruments interfaced with nano ultimate HPLC (Dionex). RP-HPLC purified peptide fractions were injected individually into the LC-MS/MS system to identify the sequences of the peptides. The peptides were analyzed using a Data-Dependent method. The acquired spectra data were searched against all influenza strains protein database using Proteome Discoverer (Thermo) to interpret data and derive peptide sequences.

Synthetic peptides were made and subjected to LC-MS/MS analysis under identical experimental conditions as described above and their sequences were confirmed based on their MS/MS data. Candidate peptide sequences were confirmed by comparison of their MS/MS spectra with that of their synthetic analogs.

Heparinized blood from healthy HLA-A2+ donors was purchased from Research Blood Components, LLC (Brighton, Mass.). Peripheral blood mononuclear cells (PBMC) were purified using differential centrifugation following standard methods. PBMC were used to generate peptide specific CTL as described previously (Testa, et al. (2012), PLoSOne in press).

Antigen stimulated interferon-γ (IFN-γ) release as a measure of CTL activation was assayed using an ELISPOT assay kit (BD-Pharmingen, San Jose, Calif.) according to the manufacturer's instructions. Results are presented as the number of interferon-y producing cells per 10⁶ PBMCs. Each assay was performed with PBMC from at least three different healthy HLA-A2+ donors. Error bars represent SEM of experimental replicates.

Ninety two epitopes including HLA-A2, A24, B7 and HLA-DR specific motifs were identified (Table 1 and 2). Almost all the peptide sequences were present in multiple strains of influenza virus family, a representative strain is shown in Table 1. Five HLA-A2 specific epitopes (SEQ ID: 1(P1), 2(P2), 3(P3), 4(P4) and 6(P5)) were selected for CTL characterization. Synthetic peptides were used for CTL analysis.

TABLE 1
List of identified influenza specific T cell epitopes, their
sequences, corresponding proteins, strains and accession ID's

| SEQ | | | Influenza | |
|---|---|---|---|---|
| ID | Peptide | Protein | strains | Accession ID |
| 1 | YINTALLNA (P1) | polymerase PA | H5N1 | gi168805480 |
| 2 | TVIKTNMI (P2) | polymerase PB1 | H5N1 | gi172053048 |
| 3 | PVAGGTSSIYI (P3) | polymerase PB2 | H3N2 | gi215480628 |
| 4 | MTIIFLILM (P4) | hemagglutinin | H2N3 | gi257123295 |
| 5 | ITFHGAKEI | Matrix protein 1 | H3N2 | Q77GW1 |
| 6 | AIMDKNIIL (p5) | nonstructural protein 1 | H1N1 | gi138898 |
| 7 | AINGITNKV | Hemagglutinin | H1N1 | gi224181223 |
| 8 | SPDDFALIVNA | polymerase PB1 | H5N1 | gi224181223 |
| 9 | EEMGITTHF | RNA-directed RNA | H1N1 | A4GCJ4 |
| | | polymerase | | |
| 10 | VETPIRNEW | Matrix protein 2 | H13N6 | Q0A415 |
| 11 | REILTKTTV | Polymerase basic protein 2 | H5N1 | O56266 |
| 12 | LPFDRTTIM | nucleocapsid protein | H3N2 | Q91UL1 |
| 13 | IIELAEKTM | polymerase PA | H5N1 | O89750 |
| 14 | IIKLLPFTA | Polymerase basic protein 2 | H5N1 | Q6DNM0 |
| 15 | ILDGVNGTLI | hemagglutinin | H3N2 | gi479034 |
| 16 | IMNKSITL | nonstructural protein 1 | H1N1 | gi89789288 |
| 17 | APPKQSRMQF | polymerase PB2 | H1N1 | Q07FH5 |
| 18 | ITNKVNTVI | Hemagglutinin | H1N1 | P03452 |
| 19 | KIQRSQDPTML | polymerase PB2 | H3N8 | gi225691060 |
| 20 | LTEKAVDSVT | neuraminidase | H5N1 | gi134037018 |
| 21 | MIGIMPDMTPSTE | Polymerase basic protein 2 | H4N2 | P26112 |
| 22 | MVIGMVSLVL | neuraminidase | H5N1 | gi169124121 |
| 23 | NANTLSSVTT | Hemagglutinin | H8N4 | P03456 |
| 24 | NIESRPQI | hemagglutinin | H3N2 | gi182341715 |
| 25 | NIVAEQGVTS | nonstructural protein 1 | H5N1 | gi118584921 |
| 26 | NTDRGVTAACP | hemagglutinin | H1N2 | gi850403785 |
| 27 | PAATALATTI | PB1 polymerase subunit | H3N2 | gi38154738 |
| 28 | PMYVGVKSL | hemagglutinin | H9N2 | gi82653835 |
| 29 | QIMPCEPTIIE | neuraminidase | H3N2 | gi134047547 |
| 30 | QVLAELKDI | Polymerase acidic protein | H7N1 | P12444 |
| 31 | RVAMPKQI | PB1-F2 protein | H1N1 | gi158958091 |
| 32 | SIAEAIIVAMV | Polymerase basic protein 2 | H4N2 | P26112 |
| 33 | STVASSLVLAV | hemagglutinin | H9N3 | gi145284498 |
| 34 | TADKRITEMIPE | Polymerase basic protein 2 | H1N1 | P03427 |
| 35 | TIIAVSNIL | hemagglutinin | H3N2 | gi218848553 |
| 36 | VIAFAIVSI | haemagglutinin | H5N2 | gi240845920 |
| 37 | VILDVILHVV | neuraminidase | H1N8 | gi324446 |
| 38 | VTIGIASLILQ | neuraminidase | H1N1 | gi216409272 |
| 39 | GTGSWPDGANINFMP | Neuraminidase | H3N2 | Q75VQ4 |
| 40 | GVSSACSYLENPSF | hemagglutinin | H5N1 | gi225697330 |
| 41 | IVWGIHHPATLKEH | hemagglutinin | H11N2 | gi238823794 |
| 42 | NTKLPFQNLSLRTVG | hemagglutinin | H10N7 | gi853877321 |
| 43 | QAAERMEVAS | Matrix protein 1 | H3N2 | Q67185 |
| 44 | ITNKVNSIVDKMNT | hemagglutinin | H5N2 | gi148532744 |
| 45 | KESDEALNMTMASTP | nonstructural protein 1 | H3N2 | gi110733621 |
| 46 | EESDEALKMSMASTP | Non-structural protein 1 | H3N2 | Q2VNE7 |
| 47 | STQAAVDQINGKLNR | Hemagglutinin | H3N2 | Q38SQ8 |
| 48 | LENERTLDF | Polymerase acidic protein | H5N2 | Q0A2I0 |
| 49 | MEAVPLITI | hemagglutinin | H9N2 | gi202071398 |
| 50 | VEQEIRTF | Nuclear export protein | H7N7 | P08275 |
| 51 | VEQELRTF | nonstructural protein 2 | H1N1 | gi156536176 |
| 52 | YPDTGKVM | Neuraminidase | H1N1 | A4U7A9 |
| 53 | YPDASKVM | neuraminidase | H1N1 | gi157168462 |
| 54 | QPETCNQSII | Neuraminidase | H1N1 | Q8QHT3 |
| 55 | VPESKRMSL | PB1 | H3N2 | H6X0A1 |
| 56 | YAFAMSRGSGSGI | hemagglutinin | H1N2 | gi852806777 |
| 57 | WLTKSDGNS | hemagglutinin | H4N2 | gi261265397 |
| 58 | VTVACPYAGA | hemagglutinin | H1N1 | gi166079429 |
| 59 | TILLVITASN | hemagglutinin | H9N2 | gi120970844 |
| 60 | TVETANIGKI | Hemagglutinin | H7N3 | P03458 |
| 61 | SQQRASAGQI | nucleocapsid protein | H3N8 | gi189230639 |
| 62 | SIFFESAGN | hemagglutinin | H5 | gi156602641 |
| 63 | SIGFYQILSI | hemagglutinin | H5N1 | gi50843950 |
| 64 | SLGIKSDAQL | Hemagglutinin | H7N7 | P26103 |
| 65 | RVSETIQRF | nonstructural protein 1 | H5N1 | gi157955890 |
| 66 | RVSETLQRF | nonstructural protein 1 | H3N8 | gi257123647 |
| 67 | RTPGNAEIEDLIFL | nucleoprotein | H5N1 | gi84797572 |
| 68 | QAVAVVNYG | Neuraminidase | H3N8 | P08326 |
| 69 | MKTISLITI | hemagglutinin | H9N2 | gi169212584 |
| 70 | LLVALENQHI | hemagglutinin | H3N8 | gi226425859 |
| 71 | LLYALLATDA | hemagglutinin | H1N2 | gi238057037 |
| 72 | LMSELGVPF | neuraminidase | H9N2 | gi215536411 |
| 73 | LNTSSRGILE | polymerase PB1 | H5N1 | gi86753744 |
| 74 | KTFFGWKD | polymerase PA | H1N2 | gi257127259 |
| 75 | KVKGQLKNN | hemagglutinin | H1N1 | gi158188098 |
| 76 | KVVKSVELN | neuraminidase | H2N1 | gi222543892 |
| 77 | KVVQSVELN | neuraminidase | H5N1 | gi47834904 |
| 78 | LANSKSQVNRQI | neuraminidase | H3N2 | gi167996878 |
| 79 | ILRDCSVGGWLLG | hemagglutinin | H5N3 | gi148532736 |
| 80 | FAWRSINED | nonstructural protein 1 | H1N1 | gi225733736 |
| 81 | DLLLKANSWS | hemagglutinin | H1N1 | gi225733727 |
| 82 | AIIVSMVFS | PB2 polymerase subunit | H9N2 | gi5732347 |
| 83 | LQSLQQIESIIEA | Polymerase acidic protein | H5N2 | Q0A2I0 |
| 84 | ETKGVTAACSYS | Hemagglutinin | H1N1 | Q9WCE1 |
| 85 | MQFGSSSEDLN | Nuclear export protein | H5N1 | P0C5T7 |

The mass spectra data were also searched against SARS coronavirus genetic database to identify epitopes shared by SARS and influenza viruses. In Table 2, epitopes that share sequences with SARS coronavirus and influenza virus are shown (SEQ ID: 86-92). Over 50% homology between the SARS and influenza sequences were observed. More importantly the sequences had MHC binding motif indicating these epitopes capability in activating a CTL response.

TABLE 2
List of T cell epitopes shared by SARS
coronavirus and Infleunza virus

| SEQ | | SARS | Influenza |
|---|---|---|---|
| ID | Peptide | Protein | protein |
| 86 | QRGAEAAVKPLLA | hypothetical | Neuraminidase, |
| | | orf14 protein | H6N1 |
| 87 | TRLQSLENV | orf lab | nuclear export |
| | | | protein, H1N1 |
| 88 | DVLSRLDKV | spike protein | Polymerase, |
| | | | PB2 H5N1 |
| 89 | LIIRENNTV | ORF1 | Neuraminidase, |
| | | | H3N8 |
| 90 | FRVVMAMFSKT | spike protein | polymerase PB1, |
| | | | H1N1 |
| 91 | PMYTVSKGTQQS | Spike | Hemagglutinin, |
| | | glycoprotein | H3N8 |
| 92 | VTDVTQLYL | nsp13-pp1ab | Hemagglutinin, |
| | | | H3N8 |

### Productive Infection With Influenza Virus Strains

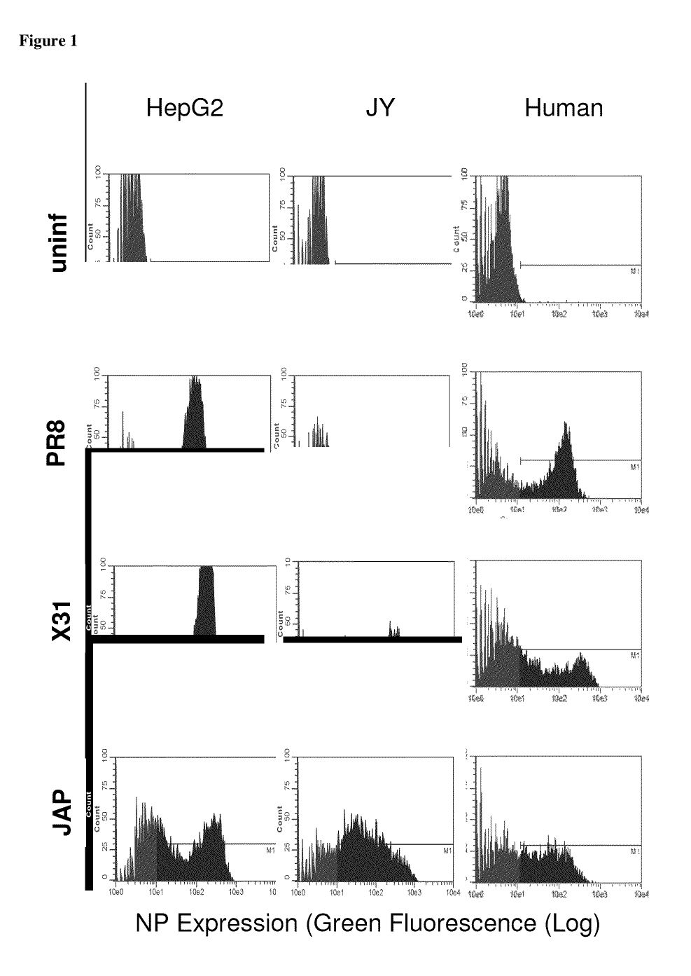

We determined the infectivity of JY, HepG2 cells and primary human DCs from HLA-A*0201+ donors, which possess high levels of the MHC class I molecule that is most globally prevalent, HLA-A molecule. We first established the infectivity of these cell types in vitro by infecting the cells with infectious viral strains A/PR/8/34(H1N1) (PR8), A X-31, Aichi/68(H3N2) (X31), or A2/Japan/305/57(H2N2) (JAP) and assessed nucleoprotein expression (FIG. 1). As demonstrated, all the three cell types were efficiently infected with various strains of virus.

### Identification of MHC Class I Presented Peptides by Nano-LC/MS/MS Analysis

MHC class I associated peptides isolated from influenza virus infected cells were subjected to LC/MS/MS analysis to identify the peptides and their corresponding proteins. Employing this strategy, we identified ninety two MHC associated peptides (Seq ID: 1-92). Prior to CTL characterization experiments, we confirmed the authenticity of the peptides and selected five HLA-A2 specific peptides (Seq ID: 1(P1), 2(P2), 3(P3), 4(P4) and 6(P5)) using their synthetic peptide analogs.

### CTLs Generated In Vitro With Influenza Epitopes are Specific and Cross-Reactive

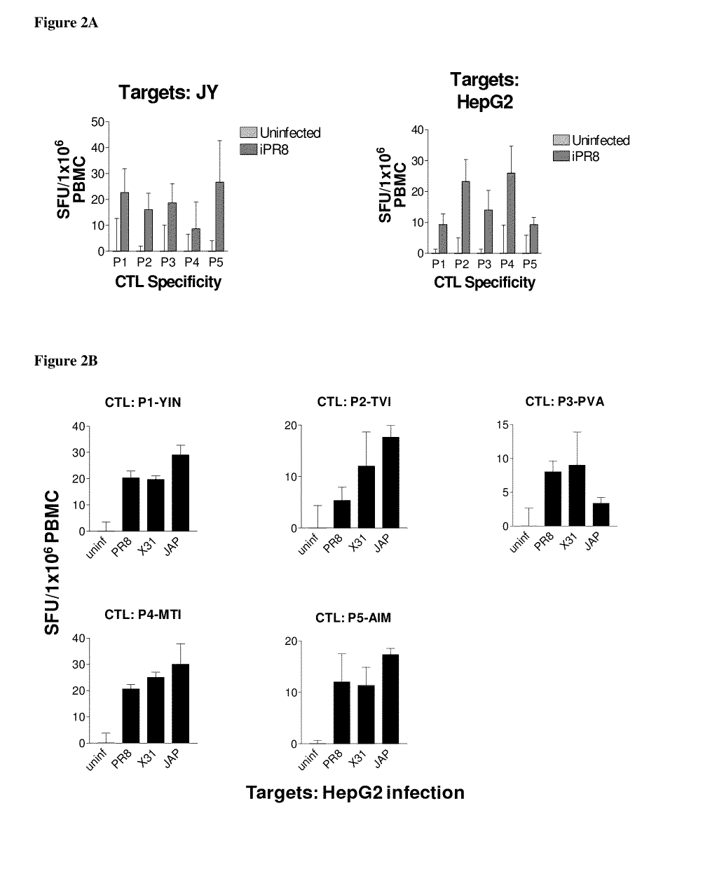

To verify the presentation of these epitopes by infected cells, CTLs specific for each of the 5 peptides were generated using PBMCs from healthy HLA-A2+ donors and synthetic peptides corresponding to the identified epitopes. In ELISpot assays, CTL functionality was measured by detection of antigen specific IFNγ secretion. As illustrated in FIG. 2A, PR8-infected JY and HepG2 cells stimulated all five of the influenza epitope-specific T cells. Additionally, cross-reactivity to other strains was demonstrated using HepG2 target cells infected with various influenza A strains (X31,

H3N2 and JAP, H2N2), indicating the presentation of these epitopes in various influenza strain-infected cells (FIG. 2B).

### CTLs Generated With Influenza Epitopes In Vivo Using Humanized Mice are Specific and Cross-Reactive

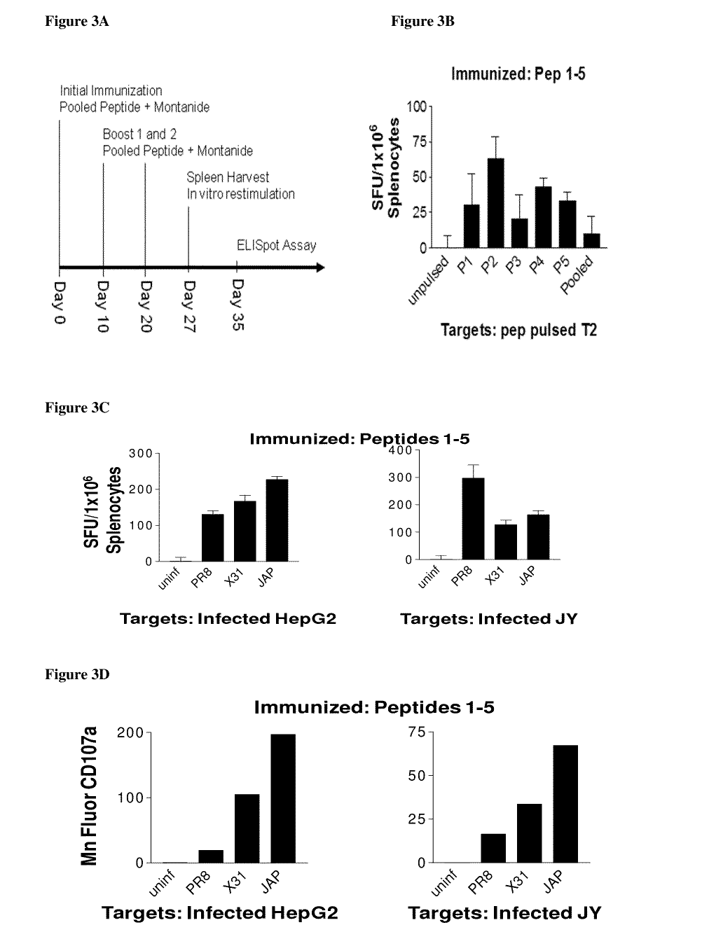

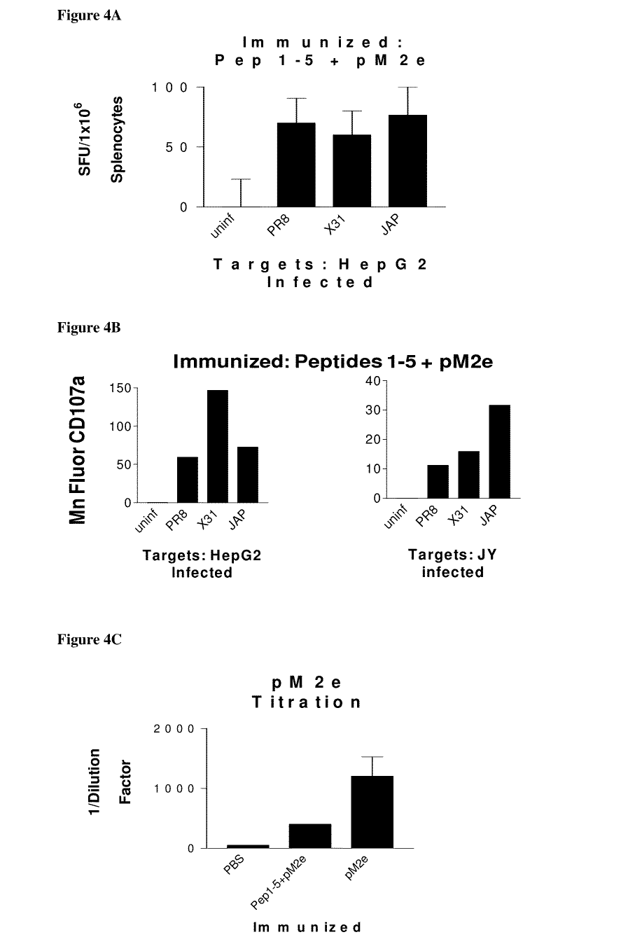

To further characterize the immune response generated by these epitopes in vivo, we immunized HLA-A2+ transgenic mice with a mixture of the aforementioned five epitopes. Immunizations were carried out using these peptides in the presence of Montanide ISA 51 as an adjuvant (FIG. 3A). We determined the influenza-specific T cell response by measuring murine IFNγ secretion in an ELISpot assay. Using T2 pulsed with individual peptides 1-5, we observed a response to all 5 peptides when mice were immunized with the mixture (FIG. 3B). In conjunction with above in vitro results, in vivo-generated CTLs specific for these peptides were stimulated equally well when HepG2 and JY cells infected with various strains of influenza were used as targets (FIG. 3C) indicating that these epitopes are presented by various influenza strain infected cells. In addition to IFNγ release, we also measured the phenotypic changes of CD8+ T cells from splenocytes with regards to CD107a, an activation marker present on granulating effector CTLs. As illustrated in FIG. 3D, splenocytes incubated with infected target cells displayed a higher staining density for CD107a when gated on CD8+ T cells.

CTLs Generated In Vivo With Influenza Epitopes in Addition to pM2e are Specific and Cross-Reactive.

Evaluation of both humoral and T cell immunity simultaneously was accomplished by the injection of multiple T cell epitopes, which drive a strong cellular response, combined with a shared antibody epitope from influenza matrix 2 protein (M2). To this end, we immunized a group of mice with MHCI peptides 1-5 in addition to a peptide from the ectodomain of M2 (pM2e). To ensure that the T cell response was at least the same as the mice immunized with only P1-5, we repeated our IFNγ ELISpot assay (FIG. 4A) and CD107a (FIG. 4B) flow cytometric analysis with the splenocytes from mice immunized with MHCI peptides 1-5+M2e peptide and observed a comparable T cell response. The concentration of circulating M2e-specific antibody was then measured by a standard ELISA using serum collected from the terminal bleeds of immunized mice. As illustrated in FIG. 4C, mice immunized with the M2e peptide generated a robust and antigen specific IgG response.

I claim:

1. An isolated peptide comprising at least one peptide having an amino acid sequence selected from the group consisting of SEQ ID NO: 1 to 92, said peptide consisting of 8 to about 20 amino acid residues, wherein said peptide binds to class I MHC molecules or processed to bind to class I MHC molecules in the activation of a T lymphocyte response.

2. A composition for use in a method of:
(a) eliciting a CTL response against influenza virus infected cells presenting at least one of the following epitopic peptides: SEQ ID NOS: 1 through 92 in a subject; or
(b) stimulating an immune response in an immunologically competent animal, said composition comprising at least one polypeptide comprising an epitopic peptide comprising an amino acid sequence selected from the group consisting of SEQ ID NOS: 1 through 92.

3. The composition for use in a method of claim 2, wherein said composition further comprises an adjuvant, optionally wherein said adjuvant is selected from the group consisting of complete Freund's adjuvant, incomplete Freund's adjuvant, Montanide ISA-51, LAG-3, aluminum phosphate, aluminum hydroxide, alum, and saponin.

4. The composition for use in a method of claim 2, wherein said composition further comprises a cytokine, optionally wherein said cytokine is selected from the group consisting of IL-1, IL-2, IL-7, IL-12, IL-15, TNF, SCF and GM-CSF.

5. The composition for use in a method of claim 2, wherein said composition further comprises a vehicle, optionally wherein said vehicle is selected from the group consisting of a liposome, nanoparticles, an immunostimulating complex (ISCOM), and slow-releasing particles.

6. The composition for use in a method of claim 5, wherein said liposome comprises an emulsion, a foam, a micelle, an insoluble monolayer, a liquid crystal, a phospholipid dispersion, or a lamellar layer.

7. The composition for use in a method of claim 2, wherein said polypeptide comprises at least one amino acid sequence selected from the group consisting of SEQ ID NOS: 1 through 92, a derivative of SEQ ID NOS: 1 through 92, and combinations thereof.

8. The composition for use in a method of claim 2(a), wherein said influenza virus infected cells are part of a viral infection.

9. The composition for use in a method of claim 8, wherein said viral infection is influenza virus A.

10. The composition for use in a method of claim 2, wherein said polypeptide comprises at least two epitopic peptides.

11. The composition for use in a method of claim 2, wherein said polypeptide comprises at least one T-cell epitopic peptide.

12. The composition for use in a method of claim 2, wherein said derivative of SEQ ID NOS: 1 through 92 and combinations thereof include a peptide comprising at least one epitopic peptide comprising an amino acid sequence having one amino acid difference from the group consisting of SEQ ID NOS: 1 through 92

13. The composition of claim 12, wherein said amino acid sequence is a T-cell epitopic peptide.

14. The composition for use in a method of claim 12, wherein said one amino acid difference is the result of a conservative amino acid substitution.

15. The composition for use in a method of claim 14, wherein said one amino acid difference is the substitution of one hydrophobic amino acid with another hydrophobic amino acid.

16. The composition for use in a method of claim 12, wherein said one amino acid difference is the addition or deletion of one amino acid to or from said epitopic peptide.

17. A method for vaccinating humans against Influenza virus comprising administering a composition containing at least one polypeptide having an amino acid sequence selected from the group consisting of SEQ ID NO: 1 through 92, a derivative of SEQ ID NOS: 1 through 92, or combination thereof.

18. A method for generating an immune response ex vivo using T cells from a subject infected with influenza virus, said method comprising: stimulating the production of CTL response for use in passive immunotherapy, wherein said T cells react with at least one polypeptide comprising an amino acid sequence selected from the group consisting of SEQ ID NO: 1 to 92; or at least one polypeptide comprising one amino acid difference from an amino acid sequence selected from the group consisting of SEQ ID NO: 1 to 92.

19. A method for assessing or diagnosing an immune response in a subject infected with influenza virus or vaccinated for influenza and related viruses said method comprising: stimulating the production of CTL response, wherein said T cells react with at least one polypeptide comprising an amino acid sequence selected from the group consisting of SEQ ID NO: 1 to 92; or at least one polypeptide comprising one amino acid difference from an amino acid sequence selected from the group consisting of SEQ ID NO: 1 to 92.
